SEQUENCE LISTING (24 sequences)

SEQ ID NO: 1 - DNA, 23 bp, Homo sapiens
tccgggctcgaagggcaaacaca

SEQ ID NO: 2 - DNA, 23 bp, Homo sapiens
cccaagaggcccacagtgaacac

SEQ ID NO: 3 - DNA, 36 bp, Homo sapiens
tgtaaaacgacggccagtcccaagggacacatcagc

SEQ ID NO: 4 - DNA, 37 bp, Homo sapiens
caggaaacagctatgacccccggcaaaggaataaact

SEQ ID NO: 5 - DNA, 39 bp, Homo sapiens
tgtaaaacgacggccagtaacctttgcagccacattttg

SEQ ID NO: 6 - DNA, 39 bp, Homo sapiens
caggaaacagctatgaccgctgctttaggggctctccac

SEQ ID NO: 7 - DNA, 39 bp, Homo sapiens
tgtaaaacgacggccagtcccccaccactaaaaatgagc

SEQ ID NO: 8 - DNA, 39 bp, Homo sapiens
caggaaacagctatgacccctggaaaagcgacacctacc

SEQ ID NO: 9 - DNA, 20 bp, Homo sapiens
tcactggctccctcattccg

SEQ ID NO: 10 - DNA, 20 bp, Homo sapiens
ctgctgtggattggattatg

SEQ ID NO: 11 - DNA, 2895 bp, Homo sapiens
agatcttactatcagtcgaaaattttgttttagccaatttgaatatgctatattgaatatgctgtagattaaatagtgaaatgtttctttggttatgctttaactatatttgagcatgcttgatattttatcaggtctctgttgtctctgatcaccaacaaggttatagactctttaaggtggaaactgacccccttcctccctttcttctctttctctcaaattctctccctattctttctctctctctctaatgcacatgtgtgtgcgctctctctctctctctgtctctcttatcctccttcctctttctttgtcttttgttcctatgttttctttatcatgaagcatacagcacagtcctacacacatatcaaacgtttgataaacactttggtgcattacactcatttgtcgagttagtcagtgtatcaaagatcacttcatgcttagtcagagttcttcccacaaatattgggtgaatacctgctctgtgcccagtgccatgctaggtcccaagggacacatcagcacatacagcaggcacatggctctgccctgtgacgctttgccctgagacatggctctgccctatgacgcttatgtcacagtggcattctacccacactttacatttccttagccctgtatattctgcagagacttttcacatatttatcttagttggttccacggcaatgctatgacttaaatatcattatttccagtttagagatgaaaaagctgaggtccagcaagtttaaattactacccccagcttatgtagctgtgtatgccacaactgaaactcaaactcaagtcttttagacttgtctaaacctttgcagccacattttgccccatccaagtgtctactggaccctgcagaataattagtttattcctttgccgggaaattcttatttcattcttcatcctatctcccgatgagtcaaatcaatgcttattcaagaattataaatcccaggtctttaggaggccgagactcgcagatcacttgaggtcagaagtttgagaccagcctggccaacatggtgaaaccctgtctctactaaaaatacaaaaattagccaggcatggtggcatgtgcctctaataccaactattccagaggctgaggcaggaaaatcccttgaacccaggaggtggaggttgcagtgaactgagatcatgccactgtactccagcctgggtgacagaatgaaatcctgtcttaaaaaaaaaaaaaaaattataacaaccccccaccactaaaaatgagcaatgccaatgggtggagagcccctaaagcagcactaattgcaaggatttctcaggtgactggttagtatgtggtgtcatatcagaaaaagaagaactgcacgaaagtatctttctgaaaacttgcaaaactgagaagctagtctgtttaaaacaggaagttatatacttacattgtttactactttactaatgtctgtgatctgatggtatctctgtttcaggagtggtgacgcctaagctatcactggacatatcaaggacttcactaaattagcaggtaccactggtcttcttgtgcttatccgggcaagaacttatcgaaatacaatagaagtttttacttagaagagattttcaggtaggtgtcgcttttccaggaaaagggaggtggaacaaggaggggtccaggcaggtggaagtagtaacttccagcagtgctcttggatctaggagggaggagaggaaggtacacagcaatttagaggtagtatggggccagagtgggagaaaatatggccacatatctccattttcttcttgctttttctaattttccctttctgtctcccatccacacaatgataagtttgcgtgtagcactcacccaaacttggccctttatacctctttagtcaacatctgtgcccatgttaccctcctatactttcacaaattctcaactatgtgcatatgactactccaaagagcttacacttaatagcttgtgtttatttatattgacttgtaagaaatagcaaaaatataattttacatatatatgtgtatatatatatatatacacatatatatatatatatatatacgcacacacacatacatttttatatatacaaagtcctccctcagtatctgtgagggatttgttccaggacccttgcagataccaaaattcatagatgctcaagttccttatataaaatgatgtagtatttgctgataacctacaaatatcctccagtaccctttaaatacaagtacctagattacttgtaatacctaatacaacgcatacagagcgcttcattcatgtggagtcaacatagcacttgctgcgtggcaaattcaaggcttggtttttgcaactttgtggagctttttcccagatatttttgatccgtgattggttgaatacacaaatgtggaacccatggatacagagggctgattatatacatatgtgtgtgtgcattaagtatgtatgtgtggaaatatatatatttatttgaagttttcattgaaaatccacactaaatcaagctactagatgagtcagttgatttgattggtgtctttttttctgggtgaatgagattgtctgtgttagagggtagagctagaaagccttaccgattcttctggaaacgcctggattgacagaggatgagtgttaactttgacaagtacttcaagtgagagtggtgggaataaggggtcttccacatggatgtggtataagttgaagtatatttttgaagtgcagctctttccagaaaatggtgatgattacactgaaccacgcaggatgagttagctcttctgggagaagaaatatgagccctttgtatgacttaataagaaaaaagcagatct

SEQ ID NO: 12 - DNA, 299 bp, Homo sapiens
aaatcccaggtctttaggaggccgagactcgcagatcacttgaggtcagaagtttgagaccagcctggccaacatggtgaaaccctgtctctactaaaaatacaaaaattagccaggcatggtggcatgtgcctctaataccaactattccagaggctgaggcaggaaaatcccttgaacccaggaggtggaggttgcagtgaactgagatcatgccactgtactccagcctgggtgacagaatgaaatcctgtcttaaaaaaaaaaaaaaaattataacaaccccccaccactaaaaa

SEQ ID NO: 13 - DNA, 11 bp, Homo sapiens
gacacatcagc

SEQ ID NO: 14 - DNA, 5 bp, Homo sapiens
ttata

SEQ ID NO: 15 - DNA, 6 bp, Homo sapiens
taaaac

SEQ ID NO: 16 - DNA, 51 bp, Homo sapiens
ggtaccactggtcttcttgtgcttatccgggcaagaacttatcgaaataca

SEQ ID NO: 17 - DNA, 22 bp, Homo sapiens
ggtaccactggtcttcttgtgc

SEQ ID NO: 18 - DNA, 22 bp, Homo sapiens
cttgtgcttatccgggcaagaa

SEQ ID NO: 19 - DNA, 21 bp, Homo sapiens
gcaagaacttatcgaaataca

SEQ ID NO: 20 - DNA, 1560 bp, Homo sapiens
agatcttactatcagtcgaaaattttgttttagccaatttgaatatgctatattgaatatgctgtagattaaatagtgaaatgtttctttggttatgctttaactatatttgagcatgcttgatattttatcaggtctctgttgtctctgatcaccaacaaggttatagactctttaaggtggaaactgacccccttcctccctttcttctctttctctcaaattctctccctattctttctctctctctctaatgcacatgtgtgtgcgctctctctctctctctgtctctcttatcctccttcctctttctttgtcttttgttcctatgttttctttatcatgaagcatacagcacagtcctacacacatatcaaacgtttgataaacactttggtgcattacactcatttgtcgagttagtcagtgtatcaaagatcacttcatgcttagtcagagttcttcccacaaatattgggtgaatacctgctctgtgcccagtgccatgctaggtcccaagggacacatcagcacatacagcaggcacatggctctgccctgtgacgctttgccctgagacatggctctgccctatgacgcttatgtcacagtggcattctacccacactttacatttccttagccctgtatattctgcagagacttttcacatatttatcttagttggttccacggcaatgctatgacttaaatatcattatttccagtttagagatgaaaaagctgaggtccagcaagtttaaattactacccccagcttatgtagctgtgtatgccacaactgaaactcaaactcaagtcttttagacttgtctaaacctttgcagccacattttgccccatccaagtgtctactggaccctgcagaataattagtttattcctttgccgggaaattcttatttcattcttcatcctatctcccgatgagtcaaatcaatgcttattcaagaattataaatcccaggtctttaggaggccgagactcgcagatcacttgaggtcagaagtttgagaccagcctggccaacatggtgaaaccctgtctctactaaaaatacaaaaattagccaggcatggtggcatgtgcctctaataccaactattccagaggctgaggcaggaaaatcccttgaacccaggaggtggaggttgcagtgaactgagatcatgccactgtactccagcctgggtgacagaatgaaatcctgtcttaaaaaaaaaaaaaaaattataacaaccccccaccactaaaaatgagcaatgccaatgggtggagagcccctaaagcagcactaattgcaaggatttctcaggtgactggttagtatgtggtgtcatatcagaaaaagaagaactgcacgaaagtatctttctgaaaacttgcaaaactgagaagctagtctgtttaaaacaggaagttatatacttacattgtttactactttactaatgtctgtgatctgatggtatctctgtttcaggagtggtgacgcctaagctatcactggacatatcaaggacttcactaaattagca

SEQ ID NO: 21 - RNA, 51 nt, Homo sapiens
gguaccacuggucuucuugugcuuauccgggcaagaacuuaucgaaauaca

SEQ ID NO: 22 - RNA, 21 nt, Homo sapiens
gguaccacuggucuucuugug

SEQ ID NO: 23 - RNA, 22 nt, Homo sapiens
cuugugcuuauccgggcaagaa

SEQ ID NO: 24 - RNA, 21 nt, Homo sapiens
gcaagaacuuaucgaaauaca

(19) United States
(12) Patent Application Publication
(10) Pub. No.: US 2002/0151064 A1
(43) Pub. Date: Oct. 17, 2002

(54) Regulation of CCR3 expression

(75) Inventors: Marc E. Rothenberg, Cincinnati, OH (US); Nives Zimmermann, Cincinnati, OH (US)

Correspondence Address: Beverly A. Lyman, Ph.D., Wood, Herron & Evans, L.L.P., 2700 Carew Tower, 441 Vine Street, Cincinnati, OH, 45202-2917, US

(73) Assignee: Children's Hospital Medical Center, Cincinnati, OH (US)

(21) Appl. No.: 10/068,067

(22) Filed: Feb. 5, 2002

Related U.S. Application Data
(60) Provisional application No. 60/267,073, filed on Feb. 7, 2001.

(51) Int. Cl. A61K 48/00; C12N 15/87

(52) U.S. Cl. 435/455; 514/44

(57) ABSTRACT

A method of regulating CCR3 expression by transcriptional and mRNA control. In one embodiment, regulation occurs in a non-promoter, regulatory region of the CCR3 gene, such as untranslated exons 1, 2, and/or 3. This type of regulation has a preferential effect on eosinophilic cells; such selectivity advantageously produces less deleterious side effects when administered in a pharmaceutical preparation. Regulation of CCR3 expression by promoter targeting also presents a method to reduce CCR3 expression in a cell-specific or nonspecific manner. Other types of regulatory compounds do not demonstrate such a preferential effect. Because CCR3 is expressed on cells involved in inflammatory reactions, regulations of CCR3 provides an intervention site for asthma, as well as other allergic, inflammatory and hypersensitivity reactions, eosinophil-mediated diseases, and infectious disorders.

### REGULATION OF CCR3 EXPRESSION

The U.S. Government has a paid-up license in this invention and the right in limited circumstances to require the patent owner to license others on reasonable terms as provided for by the terms of Grant No. Al-RO1-422420-04 awarded by the National Institutes of Health.

### FIELD OF THE INVENTION

The invention relates generally to methods for transcriptional regulation of CCR3 expression.

### BACKGROUND OF THE INVENTION

CC chemokine receptor-3 (CCR3) is a member of the family of chemokine receptors. Chemokines are chemoattractants that orchestrate leukocyte accumulation in tissue and also induce cellular activation. Chemokines are grouped into subfamilies labeled CXC, CC, C and CX₃C on the basis of the arrangement of their conserved cysteine residues. CXC chemokines are active on neutrophils, while CC chemokines have variable potencies for monocytes, lymphocytes, eosinophils, and basophils. The specific effects of chemokines are mediated by a family of seven transmembrane-spanning G-protein coupled receptors (GPCR). Seventeen of these chemokine receptors have been described: CX₃CR-1, XCR-1, CXCR-1 through 5, and CCR-1 through 10.

CC chemokine receptor-3 (CCR-3) is the major chemokine receptor expressed on eosinophils. Eosinophils are one type of granulocyte that normally appear in the peripheral blood at a concentration of about 1-3% of total leukocytes. Their presence in tissues is normally primarily restricted to the mucosa. In various disease states, eosinophils appear in increased numbers in the peripheral blood and/or tissues, a condition termed eosinophilia and described in Rothenberg, Eosinophilia, N. Engl. J. Med.1998,338:1592. Eosinophil accumulation in tissues may cause potent pro-inflammatory effects in many diseases. Eosinophilia occurs in various diseases including allergic disorders such as allergic rhinitis, asthma, and eczema, chronic inflammatory disorders such as inflammatory bowel disease, and specific syndromes such as eosinophilic gastroenteritis, eosinophilic colitis, eosinophilic cellulitis, and eosinophilic fasciitis, as well as parasitic infections and certain types of malignancies.

Because CCR3 is a critical chemokine receptor expressed on eosinophils, T cells, and inflammatory cells involved in allergic reactions (e.g., basophils), CCR3 is a potential target for intervention (treatment and/or prevention) in allergic diseases. Allergic diseases include asthma, atopic dermatitis, allergic rhinoconjunctivitis, hay fever, allergic conjunctivitis, as well as other hypersensitivity reactions such as food allergies. In addition, CCR3 is a potential target in eosinophilic hematopoietic diseases, such as eosinophilic leukemia or acute myelogenous leukemia type M4. Expression and modulation of CCR-3 is therefore a useful tool in assessing eosinophil targeting and in regulating eosinophil-mediated reactions and diseases.

CCR3 is also expressed in structural cells such as epithelial cells in lung. Because lung epithelium is a major source of inflammatory cytokines, as well as a reservoir for viral infectious agents such as respiratory syncytial virus (RSV), it is likely that CCR3 modulation will have diverse effects on inflammatory and infectious diseases. CCR3 is also expressed in endothelial cells. Additionally, CCR3 can serve as a co-receptor for the human immunodeficiency virus (HIV) and, therefore, modulation of its expression may affect the course of HIV infection.

CCR3 binds multiple ligands, defined as a protein molecule that binds to another molecule. These ligands include the polypeptides eotaxin-1, eotaxin-2, and eotaxin-3, RANTES (regulated upon activation normal T-cell expressed and secreted), and monocyte chemotactic protein (MCP)-2, MCP-3, and MCP-4. Of these chemokine ligands, only eotaxins exclusively signal through CCR-3 receptors. Previous approaches for modulating CCR3 include neutralizing the ligands for CCR3, for example, by humanized anti-eotaxin antibodies, or producing small molecule inhibitors against CCR3 (Bertrand and Ponath, Exp. Opin. Invest. Drugs 2000;9:43).

While there are numerous pharmaceutical agents that are used to treat a variety of eosinophil-related diseases, none of these agents has a mechanism of action that is directed specifically to eosinophils. For example, glucocorticoids are the most common treatment for allergic disorders, but glucocorticoids are nonspecific for eosinophils, in addition to being highly toxic. Another type of inhibitor is a non-specific adhesion molecule blocker, such as a very-late-antigen 4 (VLA-4) inhibitor. Interleukin-5 (IL-5), a chief eosinophil growth factor, is also under evaluation as a compound to target for the purpose of specifically inhibiting eosinophilia. In animal studies, blocking IL-5 by administering a humanized monoclonal antibody against IL-5 has been demonstrated to be highly effective in blocking eosinophil-mediated diseases such as asthma. Blocking the action of IL-5 would, therefore, likely reduce the symptoms of asthma. However, no clinically feasible small molecule inhibitors have been identified that inhibit IL-5. A humanized antibody against IL-5 has been shown to be safe in asthmatics, but the early clinical results have not been positive (Leckie et al., Lancet2000;356:2144. This may be due to the failure of this drug to deplete tissue eosinophil recruitment due to accessory pathways such as CCR3 (Foster et al., Immunol. Rev. 2001;179:173; Webb et al., Immunol. Cell Biol. 2001;79:165.

Methods and agents that specifically target eosinophil function and trafficking would therefore be desirable. Such approaches could be used for treatment of the wide variety of eosinophil-mediated conditions that are known. For example, pediatric asthma is an eosinophil-mediated condition whose incidence is on the rise and is now the chief diagnosis responsible for pediatric hospital admissions. Alleviation of pediatric asthma, along with the spectrum of other eosinophil-mediated conditions by an eosinophil-targeting agent, would be of tremendous benefit.

In addition, targeting CCR3 expression is likely to be beneficial for regulating other disease processes involving CCR3-bearing cells. For example, CCR3 positive T cells and basophils are involved in allergic diseases, CCR3 positive cells are infected by substrains of HIV, and CCR3 positive epithelial cells are infected by a variety of viral pathogens such as RSV.

### SUMMARY OF THE INVENTION

The invention is directed to methods of regulating the expression of the chemokine receptor CCR3. The method identifies new regulatory regions in the human CCR-3 gene or mRNA which can be targeted to regulate CCR-3 expression. Since CCR-3 is expressed on cells involved in allergic and/or inflammatory disorders, as well as on other cells such as eosinophils, these methods are useful for preventing or treating disorders involving these cells. Examples include allergic inflammatory and hypersensitivity reactions, certain types of leukemia, and certain infectious disorders involving CCR3, such as infections caused by human immunodeficiency virus (HIV) and respiratory syncytial virus (RSV).

In one embodiment, the method regulates expression of CCR3 at the level of untranslated exon 1 of a CCR3 gene or messenger RNA (mRNA). The method identifies regions containing regulatory sites at which regulatory elements i5 may be blocked, for example, by inhibitors for one or more transcription factors that bind to the gene in this region. The inhibitors prevent the binding of the transcription factors and thus prevent transcription of the CCR3 gene. Specific regulatory regions in untranslated exon 1 include nucleotides (also called base pairs (bp)) +10 to +60. These regions contain binding sites for transcription factors, such as GATA-1, GATA-2, GATA-3, C/EBP, and/or AML-1a. Additionally, antisense oligonucleotides directed against exons 1, 2, or 3 will prevent mRNA accumulation, and thus down-regulate CCR3 expression. In another embodiment, the method regulates expression of CCR3 at the level of the CCR3 promoter.

One advantage of the inventive method is the ability to selectively regulate CCR3, rather than a broad immunosuppressive approach such as glucocorticoids. Such selectivity results in less deleterious side effects in a pharmaceutical treatment for disorders involving or mediated by eosinophils.

The invention is also directed to an isolated CCR3 regulatory site comprising nucleotides (base pairs) in an untranslated exon 1 of a human CCR3 gene or mRNA capable of binding to regulatory elements. In one embodiment, the regulatory site comprises SEQ ID NO:16. In another embodiment, the regulatory site is SEQ ID NO:17, SEQ ID NO:18, and/or SEQ ID NO:19. In still another embodiment, the regulatory site comprises SEQ ID NO: 21. In yet another embodiment, the regulatory site is SEQ ID NO: 22, SEQ ID NO: 23, and/or SEQ ID NO: 24.

The invention is also directed to a method for cell selective gene expression in a human. At least one regulatory element for binding to an untranslated exon in a human cell containing a CCR3 gene or mRNA is provided to a human in a pharmaceutically acceptable formulation. The cell may be a leukocyte, for example, an eosinophil which mediates allergic and inflammatory disorders.

The invention is also directed to a method of regulating expression of CCR3 by providing an inhibitor for a CCR3 exon 1 transcription factor to a human cell containing a CCR3 receptor. The inhibitor may bind to CCR3 exon 1 at a GATA binding site in a region which may comprise SEQ ID NO:16, SEQ ID NO:17, SEQ ID NO:18, and/or SEQ ID NO:19.

The invention is also directed to an isolated CCR3 regulatory site comprising nucleotides (base pairs) in untranslated exons 2 and/or 3 of a human CCR3 gene or mRNA capable of binding to regulatory elements, and a method of regulating expression of CCR3 by providing at least one element to regulate untranslated exons 2 and/or 3 in a CCR3 gene or mRNA.

The invention is also directed to an isolated CCR3 regulatory site comprising nucleotides (base pairs) in a promoter of a human CCR3 gene or mRNA capable of binding to regulatory elements, and a method for cell selective gene expression in a human by providing at least one regulatory element for binding to a promoter in a human cell containing a CCR3 gene or mRNA. The cell may be a leukocyte, such as an eosinophil. In one embodiment, the regulatory site is SEQ ID NO:20.

The invention is also directed to an isolated complex of CCR3 exons 1, 2 and/or 3, and an antisense oligonucleotide bound to at least one nucleotide (base pair) in exons 1, 2 and/or 3, the complex blocking mRNA accumulation.

The invention is further directed to an isolated regulatory site for human CCR3 expression, the regulatory site having a sequence shown in SEQ ID NO: 16, SEQ ID NO: 17, SEQ ID NO:18, and/or SEQ ID NO:19.

These and other advantages of the invention will be apparent in light of the following figures and detailed description.

### BRIEF DESCRIPTION OF THE FIGURES

FIG. 1 shows a Northern blot analysis of CC chemokine receptor-3 (CCR3) mRNA.

FIG. 2 shows the results of 5′-rapid amplification of cDNA ends (RACE) performed on RNA isolated from purified eosinophils.

FIG. 3 is a schematic diagram of the organization of the CCR3 gene.

FIG. 4 shows the sequence of the CCR3 promoter.

FIG. 5 shows a polymorphism of the promoter in one tested individual.

FIGS. 6A-D are histograms showing activity of the CCR3 promoter in an eosinophilic cell line.

FIGS. 7A-D are histograms showing activity of the CCR3 promoter in non-eosinophilic cell lines.

FIGS. 8A-C are histograms showing activity of CCR3 promoter deletion constructs.

FIG. 9 is a schematic representation of full length and specific regions of CCR3 exon 1.

FIG. 10 is a photograph of electrophoretic mobility shift assay results demonstrating binding of transcription factors to CCR3 exon 1.

FIG. 11 is a photograph of antibody interference assay results in the presence and absence of GATA-1 antibodies.

FIGS. 12 A-B is a photograph of antibody interference assay results with multiple members of the GATA family.

### DETAILED DESCRIPTION OF THE PREFERRED EMBODIMENT

The CCR3 gene consists of four exons and spans about 23 kilobases (kb) (Zimmermann et al., Blood 2000;96:2346), which is expressly incorporated by reference herein in its entirety. The four exons give rise to multiple messenger RNA (mRNA) species through alternative splicing. The mRNAs are then translated into CCR3 protein. Previously reported sequences of the CCR3 gene have been from the region coding for the CCR3 protein (exon 4). We have sequenced regions upstream of the coding region, which reveal two putative TATA boxes and possible DNA binding sites for several transcription factors, including GATA-1, AML1, and C/EBP.

Of the four exons, exon 4 contains the open reading frame (ORF) and 11 bp of the 5′ untranslated region. Exons 1, 2 and 3 are located at the 5′ terminus and are spliced into the final mRNA. While exons 2 and 3 are present at low frequency in mRNA (<10% of transcripts), exon 1 is present in all CCR3 mRNA transcripts. Because of this, the regulatory aspects of CCR3 untranslated exon 1 were evaluated.

The materials and methods used are as follows. Hematopoietic cells were grown in RPMI 1640 (Gibco BRL, Gaithersburg MD) containing 10% fetal calf serum (FCS, Gibco BRL), 50 μmol/L 2-mercaptoethanol (Sigma, St. Louis Mo.), 0.1 mmol/L nonessential amino acids (Gibco BRL), 1 mmol/L sodium pyruvate (Sigma) and penicillin-streptomycin (Gibco BRL). The following hematopoietic cell lines were used: AML14.3D10 (eosinophilic) (kindly provided by C. C. Paul, Dayton VA Medical Center, Dayton OH); L1.2 (lymphoid; mouse B cell) (kindly provided by Paul Ponath, LeukoSite, Cambridge Mass.), Jurkat (lymphoid; human T cell), and U937 (myeloid; human myelomonocytic cell) (ATCC, Rockville Md.). The AML14.3D10 cells were further differentiated with butyric acid and IL-5 as described in Zimmermann et al., J. Immunol, 2000;1 64:1055, which is expressly incorporated by reference herein in its entirety. Nonhematopoietic cells (A549 human bronchial epithelial cells) (ATCC) were grown in Dulbecco modified Eagle medium (DMEM; Gibco BRL) supplemented with 10% FCS and penicillin-streptomycin. Eosinophils were isolated by anti-CD 16 negative selection from granulocyte preparations of healthy or atopic volunteers, as described in Zimmermann et al., J. Biol Chem. 1999;274:12611, which is expressly incorporated by reference herein in its entirety.

Total RNA was prepared using the Trizol reagent (Gibco BRL) as per the manufacturer's instructions. RNA was subjected to electrophoresis in an agarose-formaldehyde gel, transferred to Gene Screen transfer membranes (NEN, Boston Mass.) in 10× standard sodium citrate (SSC) and cross-linked by ultraviolet (UV) radiation. The chemokine receptor probe was labeled with 32p using the Klenow reaction with random priming. The open reading frame (ORF) encoding for CCR3 was amplified by the polymerase chain reaction (PCR) from human genomic DNA, subcloned into pCR2.1 (Invitrogen, Carlsbad Calif.), and the CCR3 fragment liberated by EcoRl digestion. Blots were hybridized under standard conditions and washed under high stringency (0.1×SSC, 1% sodium dodecyl sulfate (SDS) at 65° C.). Oligonucleotide probes were labeled with ³²P using T4-kinase kinase (Gibco BRL) as per the manufacturer's instructions. Membranes were hybridized with the probe in oligo hybridization buffer (6×SSC, 5×Denhardt solution, 50 mmol/L sodium phosphate buffer (pH 6.8), 0.1 mg/mL herring sperm DNA, and 1% SDS) overnight at 42° C. in 5×SSC, 0.1% SDS, then membranes were exposed to film.

The template for 5′-rapid amplification of cDNA ends (RACE) was total RNA (1 μg) isolated from human eosinophils and butyric acid/IL-5-differentiated AML14.3D10 cells. The Marathon complementary DNA (cDNA) Amplification Kit (Clontech, Palo Alto Calif.) was used for 5′-RACE as per the manufacturer's instructions. The sequences of the gene-specific primers were: primary 5′-TCC GGG CTC GM GGG CM ACA CA-3′SEQ ID NO: 1 and nested 5′-CCC MG AGG CCC ACA GTG AAC AC-3′SEQ ID NO:2. The 5′-RACE products were subcloned into pCR2.1 and sequenced (DNA Core Facility, University of Cincinnati).

Human CCR3 genomic clones were isolated by screening a phage P1 library by polymerase chain reaction (PCR) (DMPC-HFF#1; Genome Systems, St. Louis Mo.). PCR primers were chosen from the CCR3 ORF sequence and the exon 1 sequence was identified by 5′-RACE. The clone identified by the ORF primers (#427-G2) (as described in Daugherty et al., Genomics 1997;41:294, which is expressly incorporated by reference herein in its entirety), and the clone identified by the exon 1 primers (#350-B3) were used for Southern blot analysis and sequencing of the BamHI/HindIII and Bg/II fragments, respectively.

The human CCR3 promoter construct was made as follows. A 1.6-kb sequence proximal to transcription initiation site at position -1544 to +60 of exon 1 was amplified by PCR, cloned into pEGFP-1 (Clontech), and subcloned into pGL3.basic (Promega, Madison Wis.) via the Bg/II and BamHI sites. This construct is referred to as the CCR3-1.6pGL3 construct. The CCR3-1.6pGL3 construct without exon 1 was made by digesting the CCR3-1.6pGL3 construct with KpnI and ligating the insert into pGL3.basic vector linearized with KpnI, and is referred to as CCR3-1.6 (-exon 1)pGL3. The exon 1 construct was engineered by re-ligating the CCR3-1.6pGL3 construct digested with KpnI following removal of the insert and is referred to as CCR3-exonl pGL3. Deletion constructs (referred to as CCR3-0.892pGL3, CCR3-0.257pGL3, CCR3-0.222pGL3, and CCR3-0.102pGL3) were amplified by PCR, cloned into pEGFP-1 or pCR2.1, and subcloned into pGL3.basic.

Hematopoietic cells (AML14.3D10, L1.2, and Jurkat) were transfected by electroporation as described in Yamaguchi et al., J. Biol. Chem. 1994;269:19410, which is expressly incorporated by reference herein in its entirety (with kind guidance from Dr. Steven Ackerman, University of Illinois, Chicago Ill.). Briefly, 1.5×10⁷ cells were electroporated in RPMI with 0.3 μg to 15 pg plasmid DNA containing the reported construct and an appropriate amount (5 μg for Li.2 and 10 μg for AML and Jurkat) of control construct (pcDNA3.βGal) at 960 μF and 350 V for AML, 300 V for Jurkat, and 250 V for LI.2. Cells were incubated for seven hours in RPMI with 10% FCS (10 mL per electroporation) and lysates were made using 0.25 mL reporter lysis buffer (Promega) per electroporation. In other experiments, cells were transfected with Effectene (Qiagen, Valencia Calif.) as per the manufacturer's instructions. A549 and U937 cells were transfected using Effectene and lysed twenty-four hours after transfection. The pGL3.SV40 (Promega) and the promoterless pGL3.basic vectors were used as positive and negative controls, respectively. The luciferase assay was performed as per the manufacturer's instructions (Promega) using 20 μL of the cell lysate. Data were recorded with a Monolight 3010 luminometer (Analytical Luminescence Laboratory, Ann Arbor Mich.) as relative light units (RLU). βP-Galactosidase activity (from 50 μL cell lysate) was measured using o-nitrophenyl β-galacto-pyranoside (ONPG) (Sigma) as a substrate in sodium phosphate buffer for two hours at 37° C. The reaction was stopped by addition of sodium carbonate and the optical density (OD) was measured at 405 nm. All data were normalized by dividing RLU (luciferase assay) by OD (β-galactosidase assay). When the deletion constructs were compared to wild-type in the AML14.3D10 cell line, transfection efficiency was normalized by co-transfecting with the renilla luciferase vector, pRL.SV40 (Promega) and the firefly and renilla luciferase activities were determined as per the manufacturer's instructions (Dual Luciferase Reporter Assay System) (Promega).

The human CCR3 promoter and exon 1 were screened for polymorphisms by sequencing three overlapping segments amplified by PCR from genomic DNA from nineteen individuals (fifteen with severe allergic asthma and four normal controls). The diagnosis of asthma was made based on symptoms and a 12% or greater increase in forced expiratory volume in one second (FEV,) after a bronchodilator or after a two-week trial of oral corticosteroids. Asthma was classified as severe, based on the FEV₁ being below 60%, and allergic, based on a positive skin prick test (≧3 mm wheal with erythema) to one or more antigens tested (environmental antigens indigenous to the Ohio valley). Normal controls were nonallergic, nonasthmatic volunteers with a negative skin prick test to all allergens tested (excluding histamine). Informed consent was obtained from all participants in these studies.

The PCR reactions were performed with approximately 0.3 μg genomic DNA, 0.5 μmol/L each primer, 0.2 mmol/L dNTPs (Roche, Indianapolis Ind.), 1.25 U Taq Polymerase (Roche) in a total volume of 50 μL. Primer pairs were as follows and all primers had the M13 primer sequence tagged (underlined): P1 5′-(TGT AAA ACG ACG GCC AGT CCC MG GGA CAC ATC AGC) SEQ ID NO:3 and 5′-(CAG, GAA ACA GCT ATG ACC CCC GGC MA GGA ATA MC T) SEQ ID NO:4; P2 5′-(TGT MA ACG ACG GCC AGT MC CTT TGC AGC CAC ATT TTG) SEQ ID NO:5 and 5′-(CAG GAA ACA GCT ATG ACC GCT GCT TTA GGG GCT CTC CAC) SEQ ID NO:6; P3 5′-(TGT AAA ACG ACG GCC AGT CCC CCA CCA CTA AM ATG AGC) SEQ ID NO:7 and P4 5′-(CAG GAA ACA GCT ATG ACC CCT GGA AAA GCG ACA CCT ACC) SEQ ID NO:8. PCR products (420-575 bp in length) were purified (Qiagen PCR Purification Kit) and sequencing was performed on the ABI sequencer (DNA Core Facility, University of Cincinnati) using dye-primer chemistry to facilitate detection of heterozygosity. Data were analyzed using DNA Star software (DNA Star, Madison Wis.).

To characterize the promoter region, test its activity in various cells types, and delineate the role of exon 1, the promoter activity of constructs containing different lengths of the promoter and exon 1 (CCR3-1 .6(-exonl )pGL3), and a construct containing exon 1 sequence were examined in several cell types.

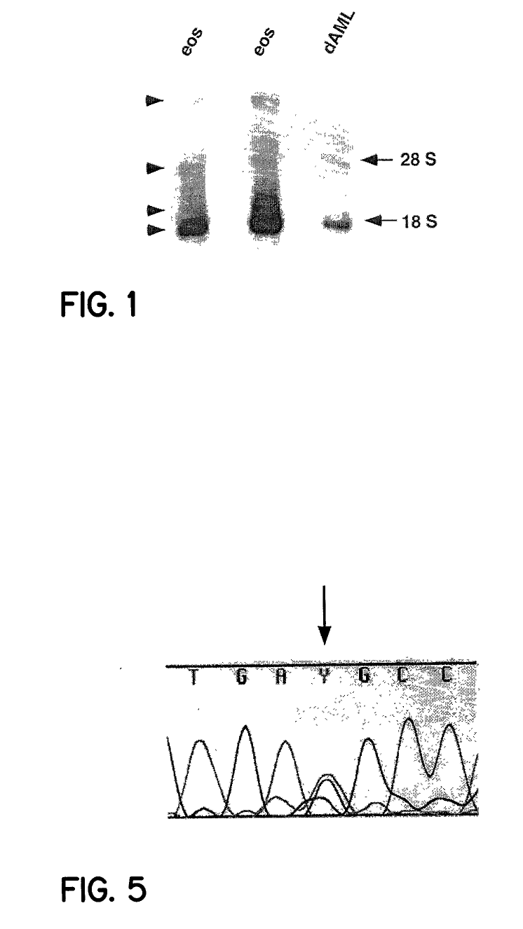

The promoters of chemoattractant receptor genes are often separated from the ORF by one or more large introns. The first evidence that this was the case for CCR3 was derived from analysis of CCR3 mRNA expression. With reference to FIG. 1, total RNA (4 μg) from the eosinophils of two individuals (eos), and from butyric acid/IL-5 differentiated AML 14.3D10 cells (dAML), was hybridized with a radiolabeled full-length CCR3 ORF probe under high stringency conditions. Autoradiography was performed for 72 hours. The locations of 28S and 18S RNA are shown and hybridized bands are labeled with an arrowhead. Northern blot analysis using a CCR3 ORF probe revealed multiple hybridizing mRNA bands. The main CCR3 mRNA migrated at ˜1.8 kb and three weaker species migrated at ˜2 kb, ˜4kb, and ˜20 kb.

The presence of multiple bands may indicate cross-hybridization with related gene products, detection of unspliced heterogeneous nuclear RNA, or the presence of multiple mature CCR3 transcripts that could arise either by alternative splicing or use of different transcription initiation or polyadenylation sites. Therefore, to characterize the CCR3 promoter, the complete sequence of the 5′ untranslated region (5′-UTR) was determined.

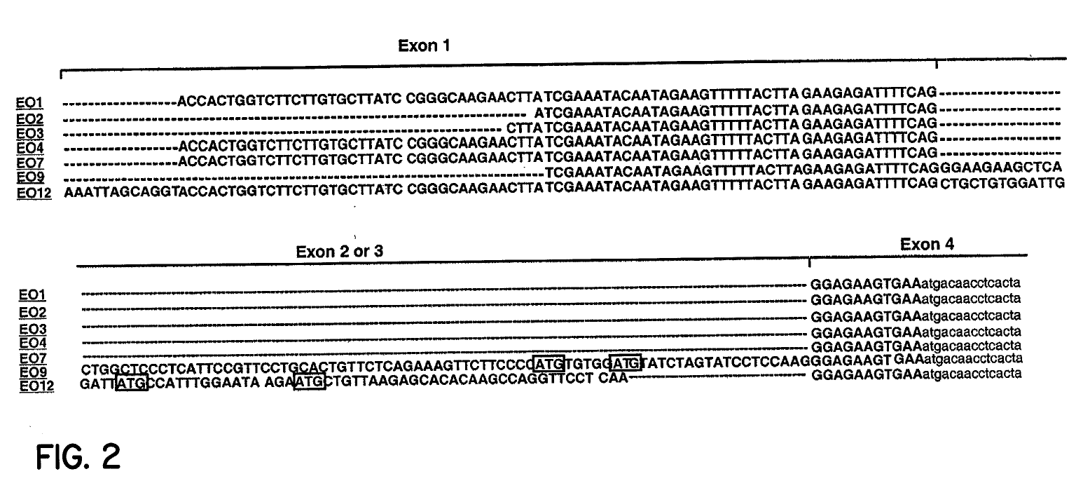

The 5′ sequence of the mature mRNA was identified by 5′-RACE using RNA isolated from eosinophils and from butyric acid/IL-5 differentiated AML14.3D10 cells. Products were subsequently subcloned and twelve clones were selected for sequencing by choosing clones with a range of insert sizes, indicated as EO with an assigned number. With reference to FIG. 2, alignment of the 5′-UTR in seven clones (labeled as EQ1, E02, E03, E04, E07, E09, and EO12) is shown in capital letters, and coding sequence as small letters. Upstream ATGs are boxed. Also in FIG. 2 the positions of exons I through 4 are indicated; —indicates a gap.

Alignment of the 5′-UTR sequence in these seven clones originally derived from eosinophil RNA revealed a complex organization. All clones had eleven bases upstream from the ATG that were identical to the genomic sequence. Additionally, all clones contained up to 93 bases of the 5′-UTR sequence that is labeled as exon 1. The truncated forms (with fewer than 93 bases) may arise from premature termination of cDNA synthesis by reverse transcriptase in vitro, or may indicate the presence of multiple transcription start sites in vivo. One 5′-RACE product had 69 bp between the two sequences (clone E012). Another clone had 89 bp between the two segments (clone E09).

These data indicate that there are three 5′ exons alternatively spliced into the final mRNA. Exon 1 is present in all transcripts, whereas either exon 2 or exon 3 is present in a small subset of mRNA species. To verify the occurrence of exons 2 and 3, all cloned 5′-RACE products were screened for their expression. Using oligonucleotide probes for each of the exons (EO9: 5′-TCA CTG GCT CCC TCA TTC CG-3′ SEQ ID NO:9 and EO1 2: 5′-CTG CTG TGG ATT GGA TTA TG-3′ SEQ ID NO:10), a low frequency of clones (<10%) containing exons 2 and 3 were identified (data not shown).

To determine the intron/exon structure of the CCR3 gene in more detail, genomic clones containing CCR3 were isolated and characterized. A genomic library was screened using PCR primers specific for the entire CCR3 ORF and exon 1. One of the clones contained the ORF and was used for Southern blot analysis and restriction map analysis. Two overlapping segments (3.8 kb BamHI and 1.7 kb HindIII fragments) were fully sequenced and shown to contain the entire ORF (located on exon 4) as well as 3591 bp of the 5′ sequence and 445 bp of the 3′ sequence. Analysis of this sequence revealed that it also contains the 69 additional bases found in 5′-RACE clone EO12, designated exon 3. Another genomic clone was identified by the exon 1 primers, and a 2.9 kb Bg/II fragment was fully sequenced and found to contain the entire exon 1. Analysis of the intron/exon junctions revealed that they conformed to the splice donor consensus sequence. Southern blotting with the exon 2 sequence (the 89 additional bp found in 5′-RACE clone E09) indicated this exon was located in the ˜8 kb BgIII-EcoRI segment.

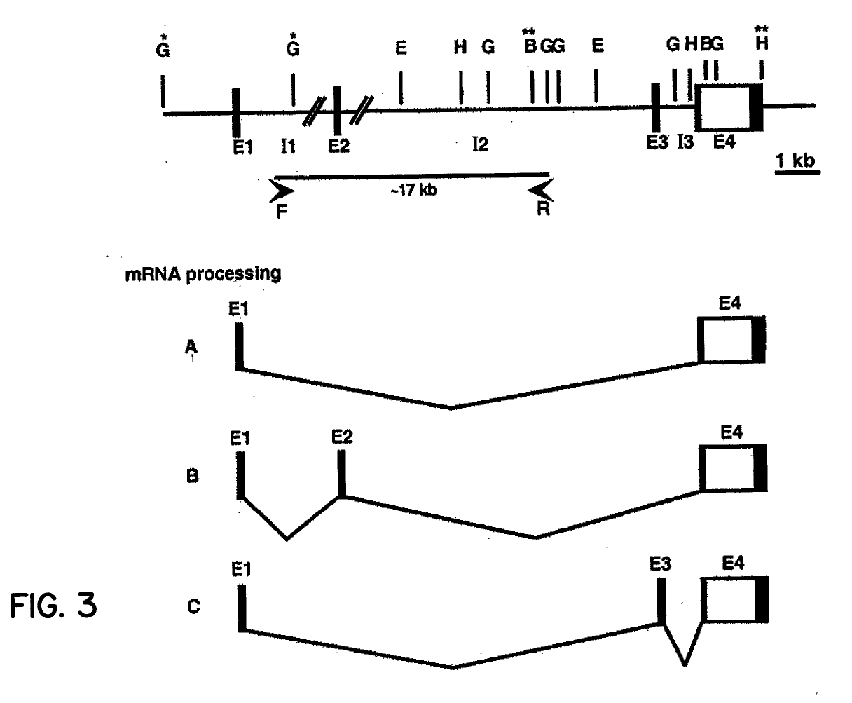

To analyze the length of the sequence intervening exons 1 and 3, long-range PCR analysis was performed using primers within the sequenced regions of introns 1 and 2 labeled as F and R, respectively. The analysis resulted in a specific band of ˜17 kb (data not shown). The proposed genomic organization and mRNA processing of the CCR3 gene is shown in FIG. 3. The translated DNA area is indicated as open rectangles and the untranslated DNA is indicated as shaded rectangles. Restriction enzymes are labeled as follows: E, EcoRI; G, Bg/II; B, BamHI; and H, HindIII. Exons are labeled as E1 through E4; introns as 11 through 13. The precise position of exon 2 has not been determined and this is indicated by the pair of slashed lines. Below are the alternative splicing mRNA species: A is the majority of mRNA species that contain only exons 1 and 4; B and C show usage of exon 1 with exons 2 or 3, respectively. DNA fragments flanked by a single asterisk (*) and double asterisk (**) were fully sequenced (Genbank accession numbers AF237380, AF237381, and U51241). Primers used for long-range PCR are labeled as F and R, flanking the ˜17 kb PCR product.

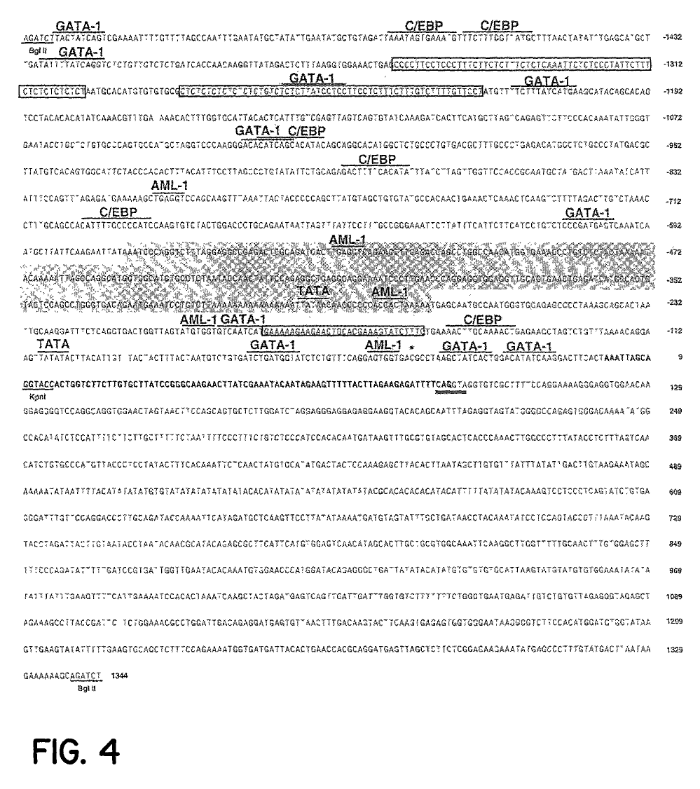

The sequence of the CCR3 promoter is shown in FIG. 4 SEQ ID NO:11. The 2.7 kb 5′ Bg/II fragment shown in FIG.3 was fully sequenced. Exon 1 SEQ ID NO:12 is bolded. The splice donor consensus sequence is double-underlined SEQ ID NO:13. Numbering is based on assigning +1 to the first base of the longest 5′-RACE product. Underlined are restriction enzyme sites (Bg/II, KpnI); overlined are consensus transcription factor binding sites; boxed are pyrimidine-rich sequences; the shaded box depicts the area of high homology with CCR2 that contains the Alu repeat, and the light gray box marks the area of homology with hlL-5Rα. The asterisk depicts the site of the identified polymorphism.

The human CCR3 promoter contained two putative TATA-boxes; one from position−298to−294 SEQ ID NO:14 and the other from position−108 to−103 SEQ ID NO:15 proximal to the first base of the longest 5′-RACE product. There are several pyrimidine (CT)-rich segments in the promoter region. For example, regions from−1361 to−1300 and from−1282 to−1224 have more than 90% C + T over more than 50 nucleotides (boxed in FIG. 4); it has been reported that CT-rich segments are present in genes abundantly expressed in myeloid cells, that is, genes for fMLP-receptor and myeloperoxidase. The promoter sequence was analyzed using the publicly available TFSEARCH engine and found to contain consensus DNA-binding sites for several transcription factors (i.e., GATA-1, AML1, C/EBP, etc.). In addition to those shown in FIG. 4, other transcription factor motifs found included AP-1, NFKB, Oct-1, CdxA, CREB, and STAT-x.

The promoter sequence was compared to other chemokine receptor promoter sequences using BestFit (SeqWeb, Genetics Computer Group, Madison Wis.). Comparison with CCR2 (accession number AF068 265) and CCR5 (accession numbers AF082 742 and AF017 632) revealed 40% overall identity (Yamamoto et al., J. Biol. Chem. 1 999;274:4646; Moriuchi et al., J. Immunol. 1997;159:5441; and McDermott et al., Lancet 1998;352:866).

However, there were areas of up to 72% identity between the human CCR3 and CCR2 promoters spanning over 200 bp (shaded area in FIG. 4). Further examination revealed that this sequence represents an Alu family repeat. Comparison to eosinophil-selective promoters (e.g., hCLC, accession number L01 665; human eosinophil peroxidase (hEPO), accession number M29 904, hIL-5Ra, accession number U18 373, and hMBP, accession number M34 462) revealed 36% to 39% overall identity (Gomolin et al., Blood 1993;82:1868;

Yamaguchi et al., J. Biol. Chem. 1994;269:19410; Sun et al., J. Biol. Chem. 1995;270:1462; and Barker et al., Gene 1990;86:285). There was a 31 bp segment with 67% homology between the CCR3 and IL-5Rcα promoter (shaded box in FIG. 4).

Several chemokine receptor promoters have been shown to be highly polymorphic and these polymorphisms are sometimes correlated with disease processes. To determine polymorphisms which may exist in the 5′-UTR and promoter of CCR3, and which may have consequences in diseases such as asthma in which the CCR3-expressing cells have a major role, genomic DNA from nineteen individuals (fifteen with severe asthma and four normal controls) was screened for single nucleotide polymorphisms using dye-primer sequencing in the first 1 kb of the promoter sequence and the entire exon 1. Only one heterozygous polymorphism was found; interestingly, this polymorphism lies in a putative CREB binding site. More specifically, DNA from one normal control individual had equal representation of cytosine and thymine bases in position−37 (FIG. 5 and asterisk in FIG. 4). Equal amounts of cytosine and thymine peaks (Y indicates thymine and cytosine) indicate heterozygosity at position−37 of the CCR3 gene (arrow). To control for PCR-introduced mutations, the same analysis was repeated using a separate PCR-amplified DNA, and the same results were obtained. These data indicate that the CCR3 promoter region and exon 1 are conserved among individuals.

Transient transfection of cell lines and reporter gene expression measurements were performed. Promoter activity results were comparable between experiments performed using the two methods. To show that the 5′ genomic region has promoter activity, 1.6 kb of 5′ DNA containing 60 bp of exon 1 was cloned into a firefly luciferase reporter vector, referred to as CCR3-1.6pGL3. The ability of the promoter construct to promote the expression of the reporter gene was first tested in an eosinophilic myelocyte cell line (AML14.3D10 cells).

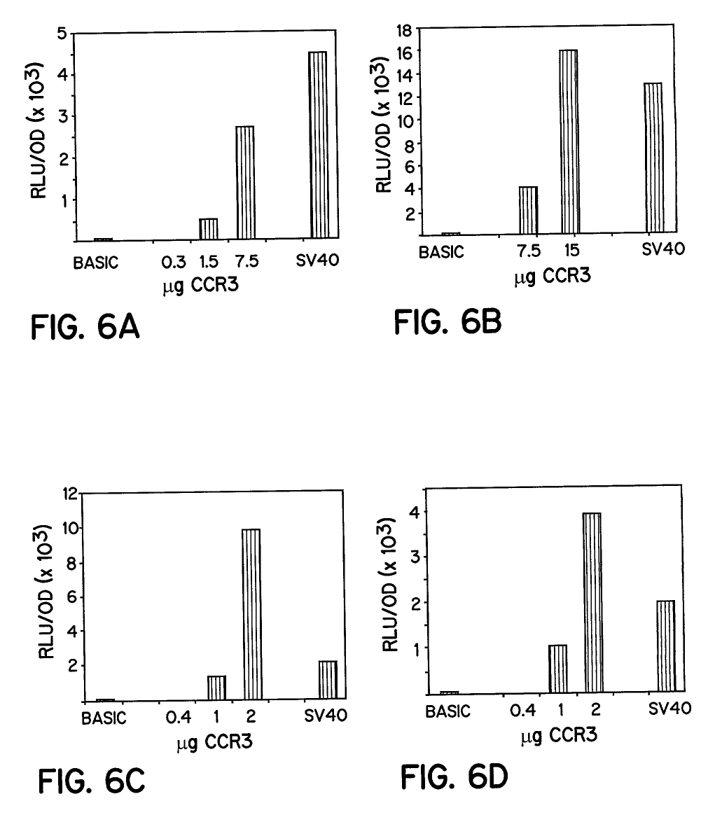

With reference to FIG. 6 A-D, AML14.3D1 0 cells were transiently transfected with a reporter plasmid containing 1.6 kb of the human CCR3 promoter, the SV40 promoter, or no promoter, ligated to the luciferase reporter gene. To correct for differences in transfection efficiency, promoter constructs were co-transfected with pcDNA3.β Gal, and data were normalized to β-galactosidase activity. The control vectors were used at 15 μg (FIGS. 6A and 6B), and at 1 μg (FIGS. 6C and 6D). On the y-axis, data are presented as RLU (luciferase activity)/OD (β-galactosidase activity).

FIGS. 6A and 6B show separate experiments where cells were transfected by electroporation. FIGS. 6C and 6D show separate experiments where cells were transfected using Effectene. Between the two methods, peak expression of the transfected proteins occurred at different times. With electroporation, expression of luciferase was about 30-fold higher seven hours after the transfection, as compared to expression at twenty-four hours. Conversely, with Effectene, expression of luciferase was about eight fold higher at twenty-four hours, as compared to expression at seven hours (data not shown).

When CCR3 promoter activity was measured at the optimal time point for each of the methods, strong expression of the luciferase gene was observed, and a dose response was seen with increased DNA concentrations. The activity of the CCR3 promoter was comparable to that of the SV40 promoter which was used as a positive control. Because of variability between experiments, two representative experiments for each of the methods are shown in FIGS. 6A-D.

When compared to the basic promoterless vector, which was used as a negative control, the CCR3 promoter activity was 45-fold higher at 7.5 μg, and 130-fold higher at 15 μg, when transfection was performed by electroporation. For comparison, SV40 promoter activity was 100-fold higher than the basic promoter at 15 μg. When transfection was performed with Effectene, the CCR3 promoter activity was 23- and 120-fold over the basic vector at 1 and 2 μg, respectively. In the same experiments, SV40 activity was 40-fold above the promoterless vector at 1 μg.

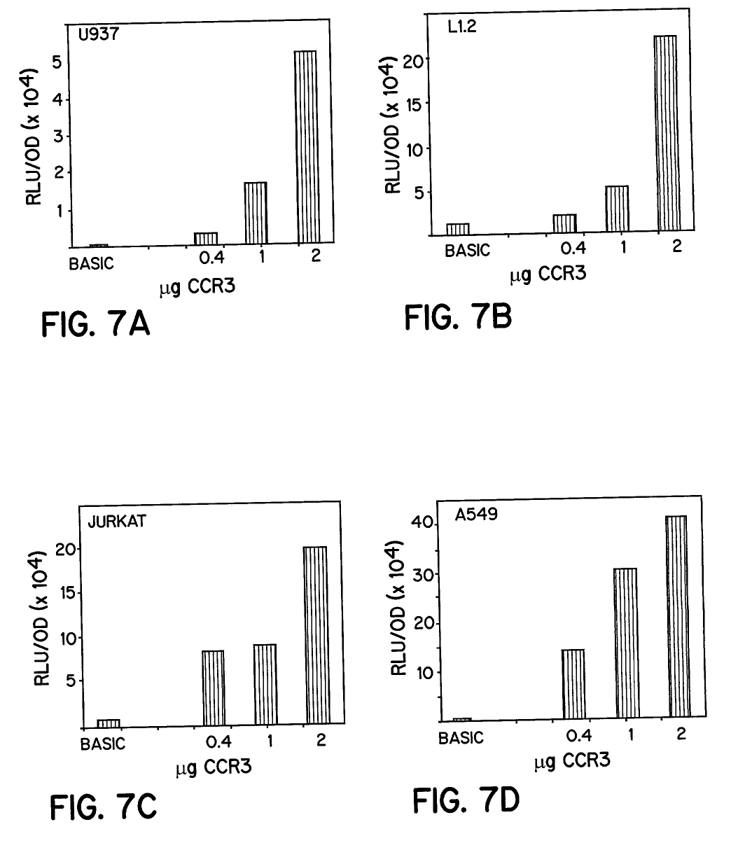

To assess whether the CCR3 promoter was specific for eosinophilic cells in vitro, the activity of the CCR3 promoter was tested in the following noneosinophilic cell lines: mouse B cell (Li.2), human T cell leukemia (Jurkat), human myelomonocytic cell (U937), and human bronchial epithelial cell (A549). Data are presented in FIGS. 7A-D for a representative experiment (n=2-4 for each cell line). Cells were transiently transfected using the Effectene method with a reporter plasmid containing either 1.6 kb of the human CCR3 promoter, or no promoter, ligated to the luciferase gene. Cells were co-transfected with the pcDNA3.βGal plasmid and the data were normalized to β-galactosidase activity. A dose response of CCR3 promoter activity is depicted, as well as the value of the promoterless vector (basic). The control vector was used at 1 μg in all cells except A549 cells, where the control vector was used at 0.4 μg. On the y-axis, data are presented as RLU (luciferase activity)/OD (β-galactosidase activity).

As shown in FIGS. 7A-D, activity in these cell lines was above the promoterless vector and was dose dependent. For example, when 1 μg of DNA was transfected using Effectene in the U937 cells, the CCR3 promoter activity was 30-fold and SV40 promoter activity was 500-fold over the promoterless vector. In the L1.2 cells, the CCR3 promoter activity was 4-fold and SV40 promoter activity was 37-fold over the promoterless vector. In the Jurkat cells, the CCR3 promoter activity was 13-fold and SV40 promoter activity was 70-fold over the promoterless vector. In the A549 cells, the CCR3 promoter activity was 56-fold and SV40 promoter activity was 2000-fold over the promoterless vector.

To localize the regulatory regions of the promoter, a series of deletion mutants were generated and tested for promoter activity. Four deletions of the CCR3-1.6pGL3 construct were prepared in the pGL3 vector. These constructs included the promoter elements starting at position−892, −257,−222, or−102 (referred to as CCR3-0.892pGL3, CCR3-0.257pGL3, CCR3-0.222pGL3, and CCR3-0.102pGL3, respectively). Deletion constructs were amplified by PCR, cloned into PEGFP-1 or pCR2.1, and subcloned into pGL3.basic. These constructs were tested for promoter activity by transiently transfecting a nonhematopoietic cell line (the respiratory epithelial line A549 cells). The A549 cells were initially chosen because the CCR3-1.6pGL3 construct displayed strong promoter activity and because transfection efficiency was highest in A549 cells compared with the other cell lines, as shown in FIG. 7D.

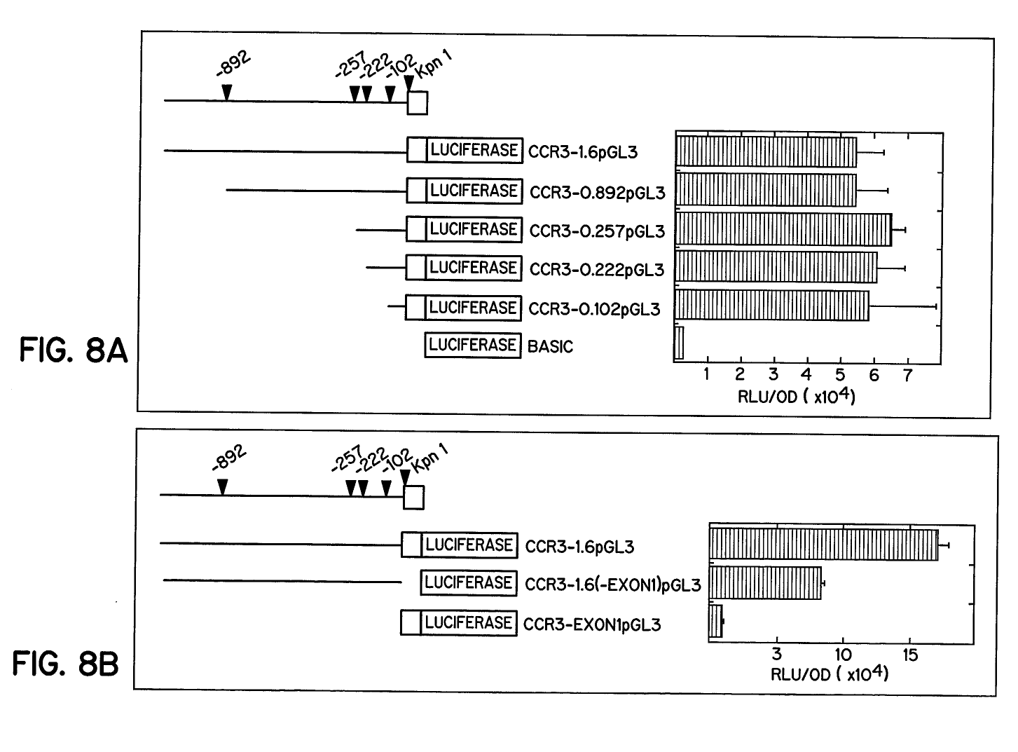

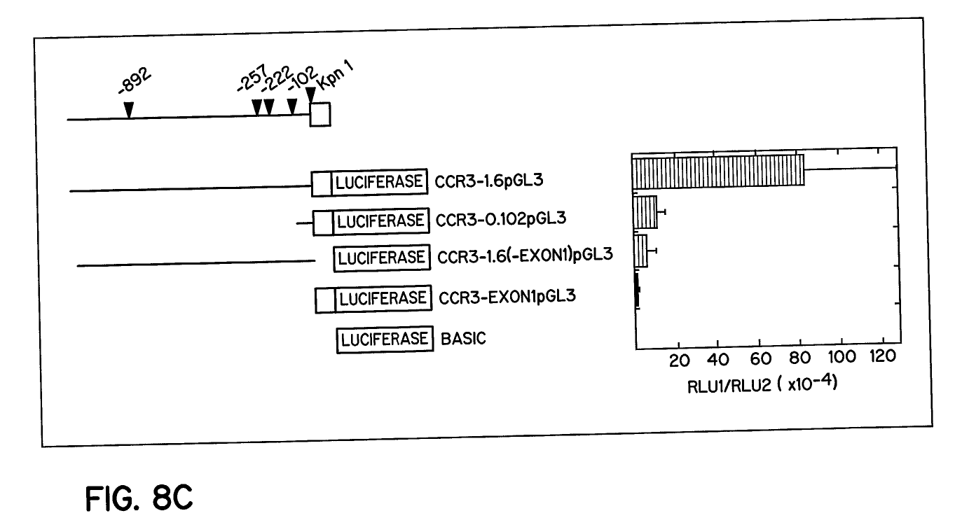

The activity of the CCR3 promoter deletion constructs was examined to delineate the role of exon 1 (FIGS. 8A-C). The promoter activity of a construct containing full promoter elements without exon 1 (referred to as CCR3-1.6(-exonl)pGL3), and a construct containing exon 1 alone (referred to as CCR3-exonl pGL3) were examined.

With reference to FIGS. 8A-C, the left panel is a schematic representation of the deletion constructs cloned into the pGL3 luciferase vector. The promoter sequence is shown as a line, and exon 1 is depicted as an open box. The position of the KpnI restriction site and the deletion construct end positions are shown with arrowheads. In the right panel, cells were transfected with the reporter plasmid indicated.

With reference to FIGS. 8A-B, A549 cells were co-transfected with the pcDNA3.β-Gal plasmid, with the data normalized to the activity of β-galactosidase. On the x-axis, data are presented as RLU (luciferase assay)/OD (β-galactosidase activity). In FIG. 8C, AML 14.3D1 0 cells were co-transfected with the pRL-SV40, with the data normalized to the activity of the renilla luciferase. On the x-axis, data are presented as RLUI (firefly luciferase activity)/RLU2 (renilla luciferase activity). Data are presented for a representative experiment (n=3). Each point was performed in triplicate and is expressed as mean±SD.

With reference to FIG. 8A, deletion of nucleotides 5′ of bp 102 did not diminish promoter activity compared to the full length vector, CCR3-1.6pGL3. Similar levels of relative promoter activity were seen at two lower doses (0.1 82 g and 0.3 μg) of the deletion constructs (data not shown). Optimal promoter activity is located within the first 102 bp of the region 5′ to exon 1.

As shown in FIG. 8B, the promoter activity was reduced by 60% ±12% (mean±SD, n=3) in the absence of exon 1. Additionally, exon 1 alone exhibited minimal promoter activity. These data indicate that exon 1 enhances the expression of CCR3, but does not act as a promoter element by itself.

After the regulatory regions of the CCR3 promoter were determined to be operational in epithelial cells, the involvement of these same regulatory regions in eosinophils was investigated. Because CCR3 is relatively eosinophil selective in vivo, it was thought that eosinophilic cells would use distinct promoter regions. The activity of the critical promoter constructs identified in respiratory epithelial cells (FIG. 8C) were therefore examined.

The CCR3-0.1 02pGL3 construct was initially examined because this region has full promoter activity in epithelial cells. In eosinophilic cells, the activity of this construct was reduced by 86.1%±11.6% (mean±SD, n=3), compared to the full length construct (CCR3-1.6pGL3). There were different promoter activity patterns between different cells types. In respiratory epithelial cells, the region with optimal promoter activity is 3′ of bp 102, whereas in eosinophilic cells, the region is 5′ of bp 102. One regulatory site includes bp-5521 to bp+9 SEQ ID NO: 20. Taken together, these results establish that the major promoter elements in eosinophilic cells are present 5′ of bp 102. In eosinophilic cells, the activity of the CCR3 construct without exon 1 was reduced by 85%±17.7% (mean±SD, n=4). However, exon 1 alone had no promoter activity, suggesting a regulatory role for untranslated exon 1. Similar to respiratory cells, maximal CCR3 transcription was dependent on exon 1, particularly in eosinophilic cells. Thus, blockade of exon 1 preferentially affects eosinophils versus other cells, but blockade of exon 1 is still effective in non-eosinophlis.

The transcription factor binding sites were identified by electrophoretic mobility shift assays (EMSA) on nuclear extracts from the eosinophilic cells (AML14.3D10 cell line). All procedures were performed at 4° C. to prevent any protease activity. Cultured cells were washed twice with ice cold phosphate buffered saline (PBS) and centrifuged. Cells were resuspended at a concentration of 2.5×10₆ cells per sample in 500 μl cold PBS and centrifuged at 10,000×g for five minutes, washed with PBS, and pelleted. The pellet was then resuspended in 20 μl of lysis buffer (100 mM 2-hydroxyethylpiperazine-N′-2-ethanesulfonic acid (HEPES), pH 7.9, 10 mM KCI, 0.1 mM EDTA, 1.5 mM MgCI_{2,} 0.2% Nonidet P-40, 1 mM dithiothreitol (DTT), and 0.5 mM phenylmethylsulfonyl fluoride (PMSF)), briefly mixed by vortex mixing, then incubated on ice for five minutes. The sample was centrifuged at 10,000×g for five minutes and the supernatant was removed and discarded. The pellet was resuspended in 20 μl extract buffer (20 mM HEPES, pH 7.9, 420 mM NaCI, 0.1 mM EDTA, 1.5 mM MgCI_{2 ,} 25% glycerol, 1 mM DTT, and 0.5 mM PMSF), briefly mixed by vortex mixing, and incubated on ice for fifteen minutes. The sample was centrifuged at 25,000×g for fifteen minutes to pellet the nuclear debris and the supernatants were placed in silicon coated microcentrifuge tubes and stored at−80° C.

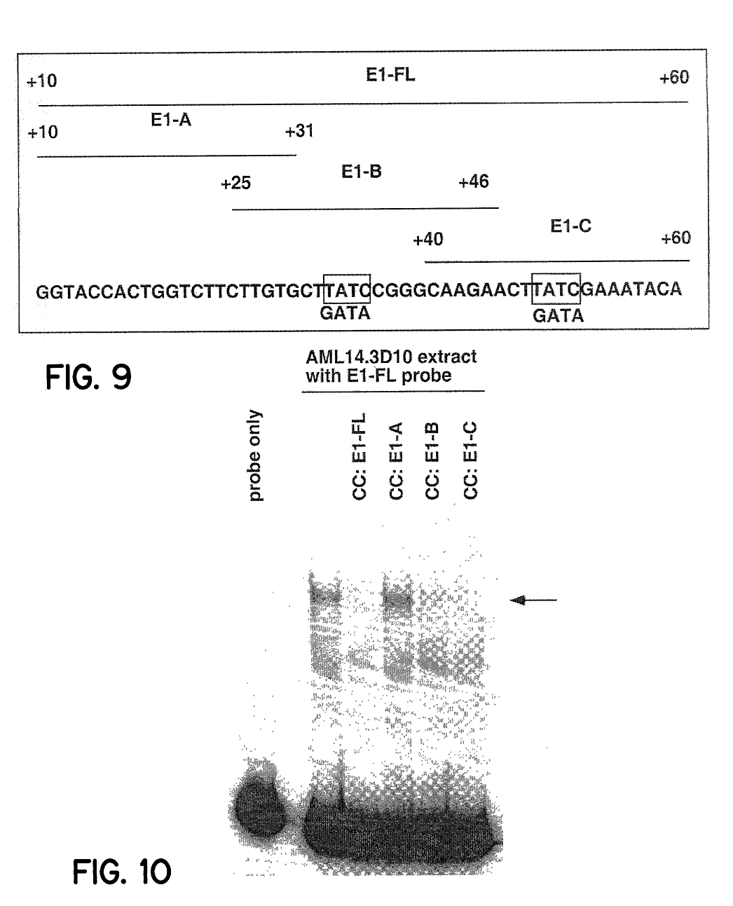

Non-radiolabeled (cold) competitor fragments of about 20 bp overlapping sequences were used to identify the specific binding area. Single-strand oligonucleotides, based on the sequence of untranslated exon 1 of the CCR3 promoter and their reverse complements, were synthesized by Integrated DNA Technologies, Inc. (Coralville Iowa.). As shown in FIG. 9, a nucleotide sequence of bp+10 to bp+60 of the exon 1 regulatory sequence for CCR3 is GGTACCACTGGTCTTCTTGTGCTTATCCGGGCAAGA ACTTATCGAAATACA SEQ ID NO:16. Transcription factor binding sites for GATA are boxed and occur at bp+33 to bp+36, and at bp+49 to bp+52, respectively. Specifically, and with reference to FIG. 9, El-FL SEQ ID NO:16 is the exon 1 full length probe. Overlapping short probes are labeled as E1-A bp+10 to bp+31 SEQ ID NO:17; E1-B bp+25 to bp+46 SEQ ID NO:18; and E1-C bp +40 to bp +60 SEQ ID NO:19, respectively. Two of the overlapping fragments, E1-A SEQ ID NO:17 and E1-B SEQ ID NO:18, contained putative GATA binding sites. Two mutants and their complements were also made which changed the GATA sequence TATC to TTGA. Corresponding regulatory sites in CCR3 mRNA are SEQ ID NO: 21 for full length exon 1; SEQ ID NO:22 for bp+10 to+31; SEQ ID NO: 23 for bp+25 to+46; and SEQ ID NO: 24 for bp+40 to+60. Each oligonucleotide was resuspended to a concentration of 50 pM in TE buffer (10 mM Tris-HCl, pH 7.4, and 0.1 mM EDTA). As cold competitors, the full length probe (bp+10 to bp+60 SEQ ID NO:16), as well as the short probes (bp+10 to bp+31 SEQ ID NO:17, bp+25 to bp+46 SEQ ID NO:18, and bp+40 to bp+60 SEQ ID NO:19) were used.

Complimentary single-stranded oligonucleotides were annealed at a concentration of 10 mM in restriction enzyme buffer M (10 mM Tris-HCl, 10 mM MgCI₂, 50 mM NaCI, and 1 mM DTT). Samples were placed in a 95° C. dry heat block for five minutes and then the block was removed from the unit and allowed to cool slowly to room temperature. The double-stranded oligomers were diluted to 1 μl with TE buffer and 30 ng were end-labeled using [P]ATP and T4 polynucleotide kinase. Probes were purified over Quick Spin G-25 Sephadex columns and recovered in a volume of 50 μl.

Protein content of the nuclear extracts was determined by the Bradford assay. Total protein (5 μg) in 12.5 μl TE was incubated on ice for ten minutes with 12.5 μl 12×EMSA buffer (24% glycerol, 0.08 μg/ml poly dl-dC, 24 mM HEPES (pH 7.9),8 mM Tris-HCl (pH 7.9),2 mM EDTA, 2 mM DTT, 50 mM KCI, and 10 mM MgCI₂) and, when indicated, with 150 fold excess of cold competitor oligonucleotide. Radiolabeled oligo probe was added to each sample and incubation continued for an additional ten minutes on ice. For antibody supershift/interference assays, 2 μl of anti-GATA-1, -2, or -3 antibody (Santa Cruz Biotechnology, Santa Cruz Calif.) was added following the addition of the probe, and samples were incubated on ice for one hour. The DNA-protein complexes were then resolved on a non-denaturing 5% acrylamide gel (29:1 acrylamide/bis-acrylamide, 0.5×TBE buffer (44.5 mM Tris, 44.5 mM borate, and 1 mM EDTA), and 25% glycerol) at a constant current of 30 mA for approximately sixty minutes. Gels were dried on blotting paper and exposed to x-ray film.

As shown in FIG. 10, the results of the EMSA without El -FL SEQ ID NO: 16, EIA SEQ ID NO: 17, and E1C SEQ ID NO: 19 show a specific band, and indicate that a protein present in the nucleus of the eosinophilic cells bound to the exon 1 sequence. Two protein binding sites were found: one in the region of bp+25 to bp+46 SEQ ID NO:18, and one in the region of bp+40 to bp+60 SEQ ID NO:19.

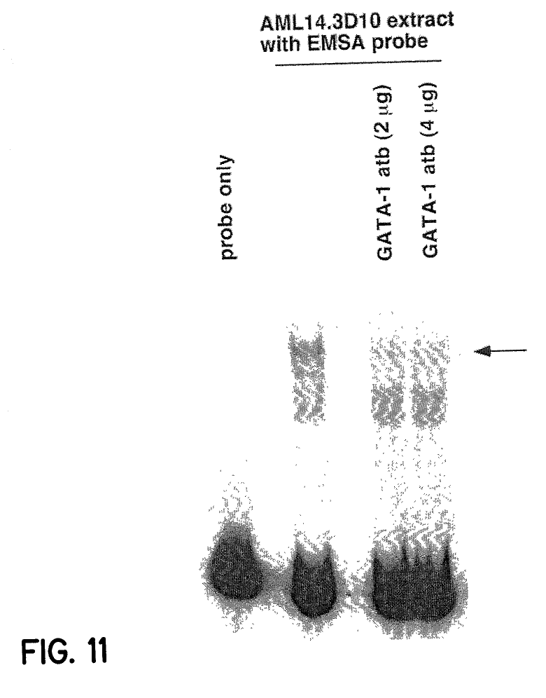

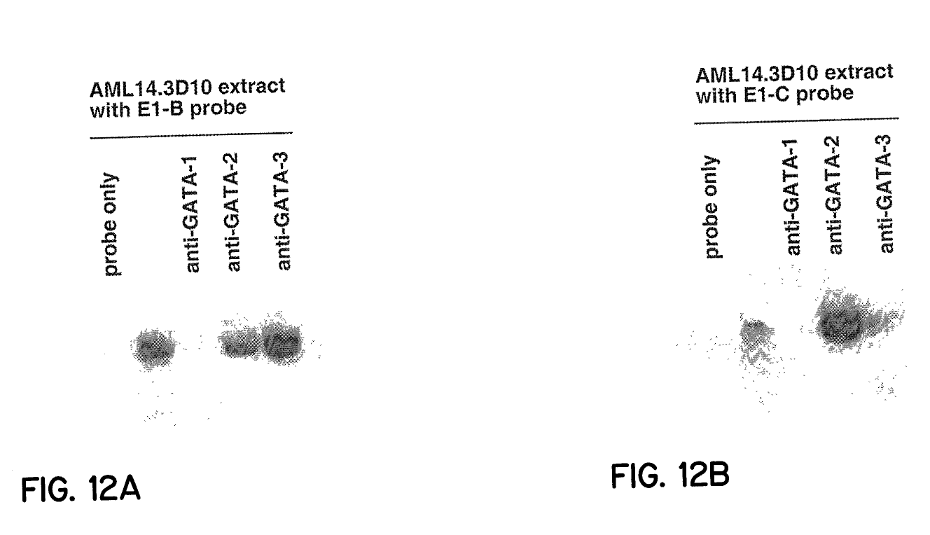

The results of antibody interference assays are shown in FIGS. 11 and 12A-B. With reference to FIG. 11, results of the EMSA with the full length probe using extracts from eosinophilic cells (AML14.3D10 cell line) in the presence or absence of antibodies to GATA-1 are shown. Preincubation of nuclear extracts with antibody to GATA-1 diminished protein binding to the exon 1 probe, demonstrating GATA-1 binding to exon 1. With reference to FIGS. 12A-B, results of the EMSA with the short probes El-B SEQ ID NO:18 and El-C SEQ ID NO:l 9 using extracts from eosinophilic cells (AML14.3D1 0 cell line) are shown. Antibody interference was performed using antibodies to GATA-1, GATA-2, and GATA-3. GATA-1 and GATA-2 bound at bp+25 to bp+46 SEQ ID NO:18. GATA-1 and GATA-3 bound at bp+40 to bp+60 SEQ ID NO:19. Other transcription factors, possibly AML-la, bound at bp+10 to bp+31 SEQ ID NO:17.

The invention has applications for the treatment of a variety of eosinophil-associated disorders. These disorders include, but are not limited to, allergies including asthma, hay fever, urticaria, eczema, favism, arachnidism, insect bites and wasp stings, reactions to foreign proteins and angioneurotic edema, eosinophilic cardiomyopathy, eosinophilic gastroenteritis, hypereosinophilic syndrome, graft versus host disease, chronic fibrosis, parasitic inflammatory disorders such as trichinosis, visceral larva migrans and strongyloidiosis, drug reactions, eosinophilic pneumonias, episodic angioedema with eosinophilia, inflammatory bowel disease, diseases of blood forming organs such as chronic granulocytic leukemia, eosinophilic leukemia, polycythemia vera, heavy chain disease, after splenectomy, collagen diseases such as polyarteritis and lupus erythematosus, food enteropathy, skin diseases such as dermatitis, contact hypersensitivity, eczema, psoriasis, pemphigus, scabies and erythema multiforme, viral infectious disorders such as HIV, RSV, and so on. A pharmaceutically acceptable formulation of at least one regulatory element for binding to an untranslated exon in a human cell containing a CCR3 gene or mRNA is administered either alone or with another drug, such as an anti-sense oligonucleotide against IL-5 or another cytokine, and/or a humanized anti-IL-5 antibody.

The CCR3 inhibitor may be administered in a pharmaceutically acceptable formulation by a variety of methods. Methods include parenteral, enteral, sublingual, ocular, nasal, or cutaneous administration, depending upon the condition to be treated and the preferred delivery vehicle. Gene delivery via mechanical, ultrasound, thermal, and/or physical applications are also included, as known to one skilled in the art. Delivery vehicles include sterile water, physiological saline or mixtures thereof, buffers, sugars, starches, lipid containing transfection or gene delivery agents, etc., as is known to one of skill in the art.

Other variations or embodiments of the invention will also be apparent to one of ordinary skill in the art from the above description. Thus, the forgoing embodiments are not to be construed as limiting the scope of the invention.

What is claimed is:

1. An isolated CCR3 regulatory site comprising base pairs in an untranslated exon 1 of a human CCR3 gene or mRNA capable of binding to regulatory elements.

2. The regulatory site of claim 1 comprising SEQ ID NO:16.

3. The regulatory site of claim 1 wherein the regulatory elements are binding sites for antisense oligonucleotides.

4. The regulatory site of claim 1 wherein the regulatory elements are binding sites for transcription factors.

5. The regulatory site of claim 4 wherein the transcription factors are at least one of GATA-1, GATA-2, GATA-3, AML-1a, and combinations thereof.

6. The regulatory site of claim 4 wherein said sites are at least one of SEQ ID NO:17, SEQ ID NO:18, and SEQ ID NO:19.

7. The regulatory site of claim 1 comprising SEQ ID NO:21.

8. The regulatory site of claim 3 wherein said sites are at least one of SEQ ID NO:22, SEQ ID NO:23, and SEQ ID NO:24.

9. A method for cell selective gene expression in a human comprising providing to said human a pharmaceutically acceptable formulation of at least one regulatory element for binding to an untranslated exon in a human cell containing a CCR3 gene or mRNA.

10. The method of claim 9 wherein said element regulates transcription of at least one of exon 1, exon 2, and exon 3.

11. The method of claim 9 wherein said element regulates binding of a transcription factor.

12. The method of claim 9 wherein said element is selected from the group consisting of a transcription factor inhibitor, an antisense oligonucleotide, and combinations thereof.

13. The method of claim 12 wherein said element is an inhibitor for a GATA transcription factor.

14. The method of claim 12 wherein the cell is selected from the group consisting of a leukocyte, an epithelial cell, an endothelial cell, and combinations thereof.

15. A method of regulating expression of CCR3 comprising providing an inhibitor for a CCR3 exon 1 transcription factor to a human cell containing a CCR3 receptor.

16. The method of claim 15 wherein the inhibitor binds to CCR3 exon 1 at a GATA binding site.

17. The method of claim 16 wherein the binding site comprises SEQ ID NO:16.

18. The method of claim 16 wherein the binding site is at least one of SEQ ID NO:17, SEQ ID NO:18, and SEQ ID NO:19.

19. An isolated CCR3 regulatory site comprising base pairs in untranslated exon 2 of a human CCR3 gene or mRNA capable of binding to regulatory elements.

20. The regulatory site of claim 19 wherein the regulatory elements are selected from the group consisting of binding sites for antisense oligonucleotides, binding sites for transcription factors, and combinations thereof.

21. A method of regulating expression of CCR3 comprising providing at least one element to regulate untranslated exon 2 in a CCR3 gene or mRNA.

22. The method of claim 21 wherein said element regulates transcription of untranslated exon 2.

23. The method of claim 21 wherein said element regulates binding of a transcription factor.

24. The method of claim 21 wherein said element is selected from the group consisting of a transcription factor inhibitor, an antisense oligonucleotide, and combinations thereof.

25. An isolated CCR3 regulatory site comprising base pairs in untranslated exon 3 of a CCR3 gene or mRNA capable of binding to regulatory elements.

26. The regulatory site of claim 25 wherein the regulatory elements are selected from the group consisting of binding sites for antisense oligonucleotides, binding sites for transcription factors, and combinations thereof.

27. A method of regulating expression of CCR3 comprising providing at least one element to regulate untranslated exon 3 in a CCR3 gene or mRNA.

28. The method of claim 27 wherein said element regulates transcription of untranslated exon 3.

29. The method of claim 27 wherein said element regulates binding of a transcription factor.

30. The method of claim 27 wherein said element is selected from the group consisting of a transcription factor inhibitor, an antisense oligonucleotide, and combinations thereof.

31. An isolated CCR3 regulatory site comprising base pairs in a promoter of a human CCR3 gene or mRNA capable of binding to regulatory elements.

32. The regulatory site of claim 31 comprising SEQ ID NO:20.

33. The regulatory site of claim 31 wherein the regulatory elements are selected from the group consisting of binding sites for antisense oligonucleotides, binding sites for transcription factors, and combinations thereof.

34. The regulatory site of claim 33 wherein the transcription factors are selected from the group consisting of GATA-1, GATA-2, GATA-3, AML-1, C/EBP, and combinations thereof.

35. A method for cell selective gene expression in a human comprising providing to said human at least one regulatory element for binding to a promoter in a human cell containing a CCR3 gene or mRNA.

36. The method of claim 35 wherein said element regulates transcription of at least one of exon 1, exon 2, exon 3, and exon 4.

37. The method of claim 35 wherein said element regulates binding of a transcription factor.

38. The method of claim 35 wherein said element is selected from the group consisting of a transcription factor inhibitor, an antisense oligonucleotide, and combinations thereof.

39. The method of claim 38 wherein said element is an inhibitor for a transcription factor selected from the group consisting of a GATA transcription factor, a C/EBP transcription factor, an AML-1 transcription factor, and combinations thereof.

40. An isolated complex of CCR3 exon 2 and an antisense oligonucleotide bound to at least one base pair in exon 2, said complex blocking mRNA accumulation.

41. An isolated complex of CCR3 exon 3 and an antisense oligonucleotide bound to at least one base pair in exon 3, said complex blocking mRNA accumulation.

42. An isolated nucleic acid regulatory site for human CCR3 expression comprising SEQ ID NO: 16.

43. The regulatory site of claim 42 selected from the group consisting of SEQ ID NO: 17, SEQ ID NO:18, SEQ ID NO:19, and combinations thereof.

44. An isolated nucleic acid regulatory site for human CCR3 expression comprising SEQ ID NO:21.

45. The regulatory site of claim 44 selected from the group consisting of SEQ ID NO: 22, SEQ ID NO:23, SEQ ID NO:24, and combinations thereof.

46. A method for cell selective gene expression in a human comprising providing to said human a pharmaceutically acceptable formulation of at least one regulatory element for binding to an untranslated exon in a human cell containing a CCR3 gene or mRNA and an agent selected from the group consisting of an anti- sense oligonucleotide against IL-5, a humanized anti-IL-5 antibody, and combinations thereof.
